(19) United States
(12) Patent Application Publication
(10) Pub. No.: US 2021/0395652 A1
(43) Pub. Date: Dec. 23, 2021

(54) TEXTILE TREATMENT COMPOSITIONS

(71) Applicants: Bruce Smyth, Byron Bay (AU); Michael Bralkowski, Lexington, NC (US)

(72) Inventors: Bruce Smyth, Byron Bay (AU); Michael Bralkowski, Lexington, NC (US)

(21) Appl. No.: 17/347,537

(22) Filed: Jun. 14, 2021

Related U.S. Application Data
(60) Provisional application No. 63/038,635, filed on Jun. 12, 2020.

(51) Int. Cl. C11D 3/48; A01N 55/00; A01N 25/10; A01N 25/08; C11D 3/00; C11D 3/32; C11D 3/30; C11D 11/00; A62B 23/02; A61L 15/44

(52) U.S. Cl. CPC C11D 3/48; A01N 55/00; A01N 25/10; A01N 25/08; C11D 3/0015; A61L 2300/208; C11D 3/30; C11D 11/0017; A62B 23/025; A61L 15/44; A61L 2300/404; C11D 3/32

(57) ABSTRACT

A novel liquid composition that provides antimicrobial and antiviral disinfection containing a quaternary ammonium salt fabric softener and a quaternary silicon compound which can be applied in a washing machine during the rinse cycle. The mixture is efficacious against most bacteria, virus and micro-organism. The dispensing in the wash cycle and machine drying sets the mixture into the fabric for residual efficacy. The compounds provide for softening and deodorizing of textiles garments. In addition, the compounds provide for sanitizing and extended residual sanitized efficacy for textiles garments. The compounds provide efficacy against many common virus, bacteria, including MRSA.

This application claims the priority benefit under 35 U.S.C. section 119 of U.S. Provisional Patent Application No. 63/038,635 entitled “Textile Treatment Compositions” filed on Jun. 12, 2020; and which is in its entirety herein incorporated by reference.

### FIELD OF THE INVENTION

The present invention relates to a chemical treatment that may be applied to a protective article. In particular, the invention relates to material compositions for controlling the spread of pathogens and infection diseases.

The present invention relates to antimicrobial and antiviral compositions, a method of manufacturing or treating a textile material, such as woven and non-woven textiles, yarn and/or fiber, with antimicrobial and antiviral compounds in such a manner to chemically bind or attach said compounds to the textile material, and to the treated textile material which performs as a disinfectant or sterilizer on its own. The treated textile material exhibits wash-durability and non-leaching properties, and can be provided with multifunctional enhancements, such as hydrophobic properties on the surface.

The present invention is also directed to a textile material to which one or more antimicrobial or antiviral agents are adhered. The agent(s) is/are adhered to the textile material in such a manner that they are not released from the textile even if the textile is wetted or washed, so that the textile is reusable. Washability and/or usability of the textile are improved where one or more agents are adhered to the textile. The textile material can be used, e.g., in a reusable textile product. The invention further relates to a method of finishing a textile material by applying and binding antimicrobial and/or antiviral agents to the textile material so that the agents are essentially irreversibly adhered to the finished textile material.

### BACKGROUND OF THE INVENTION

Disinfection/sterilization is a very important process in everyday life. It is rated at various levels. There are various recordings of the requirements of the levels of performance which can be noted, for example, as per the United States National Pesticide Information Center. A table therefrom, as can be taken hereinafter, shows that there are three main types of public health antimicrobial pesticides. The difference between the three groups is significant in terms of capability of antimicrobial and antiviral activity.

Current disinfectants available on the market work for the moment when applied or used, but are not continuous or long lasting in nature. Hence, when chlorhexidine is sprayed on a contaminated surface, it is sanitized for that instant, but as soon as the chemical is evaporated or wiped off, the surface is once again contaminable. When water is decontaminated using chlorine for example, additional amounts of water would need additional amounts of chlorine, hence requiring reusable resources.

| | Sanitizer | Disinfectant | Sterilizer |
|---|---|---|---|
| Effective against | 99.9% | 100% | 100% |
| | Bacteria | Bacteria, fungi, | Bacteria, Fungi, |
| | | viruses | Viruses and |
| | | | Spores |
| Time required for | 30 seconds to 5 | Generally 10 | Variable |
| effectiveness | minutes | minutes | |
| Location/Uses | Household | Household | Medical |
| | surfaces, | surfaces, | instruments |
| | Food contact | Medical | Research |
| | surfaces | Settings | supplies |
| Effect | Limited | Microbiocide | Microbiocide |
| | microbiocide | Irreversible | |

Textile materials like woven and non-woven fabrics, yarns and/or fibers are used for a variety of purposes and in a variety of environments. As such, there is a realistic danger of microbiological contamination on the textile surfaces. These substrates are used to filter air or water, but work only by blocking, and do not eliminate the contamination. In recent times, studies have shown that textiles carry nosocomial infections from patient to patient in hospitals. Soldiers often wear clothes for extended periods of time, without washing, which often results in fungal and bacterial infection to the wearer.

Danger of staining of apparel due to ketchup, honey, sputum, blood, human excreta and moisture are also problems faced by users in various circumstances. Not only do such stains look unpleasant, but they also are fertile breeding grounds for various harmful bacteria, fungi and viruses on the textile substrates.

When used as wearing apparel, the inner surface of the textile, dead tissue, sweat, humidity and moisture aids the growth and spread of various pathogens. Garments such as jackets and overcoats, which directly do not come in contact with the skin, are also susceptible to infection transfer through contact with the inner garments, which are possibly infected. As such, it is evident that textile contamination by microbiological pathogens is a major cause for concern.

Security and military personnel, flight attendants and other airline personnel are especially prone to disease and skin problems as they may have to wear the same clothing for more than one day. Military personnel may have to wear their apparel for as much as 28 days at a stretch. Not only can the soiled apparel cause health problems to the wearer, but also it can be breeding grounds for the spread of bacteria, fungi and virus based diseases.

In hospitals, the presence of microbes and viruses is far more threatening. Due to the nature of the environment in which textiles are used, the needs of these textiles are much more specialized. Apart from the regular textiles worn by doctors, nurses, patients and other personnel in hospitals, doctor's clinics and other such locations, textiles used in the form of scrubs, gowns, lab coats, bed sheets and pillow cases carry microbes in various proportions. Patients sleep on sheets and pillow cases that have extremely high risk of contamination due to bacterial and microbial growth resulting from excretions of the body. The mattresses and pillows are also likely to become infected due to the fact that these are not washed. They, in turn, can transmit infection to the patient. Sheets, pillow covers, gowns, and curtains are subjected to contamination from open wounds and other medical conditions, such as coughing, wheezing, etc. Patients' gowns are contaminated by sweat and/or human excretion such as urine, stool and vomit. This leads to the growth of microorganisms like bacteria, viruses and fungi. Healthcare workers are very often subjected to the contamination either from soiled textiles used by patients or due to excretions of the body. Medical personnel are major causes of transmitting bacterial infection from one patient to another. Current medical textiles offer no barrier protection.

Provided herein below are current situations and problems thereof in hospitals:

a. Hospital or healthcare transmitted diseases to a great extent are textile based transmissions.
b. Doctors and patients tend to infect each other through textile contact.
c. Current methods of washing lead to damage of the textile.
d. Pillows, mattresses and curtains are rarely washed or disinfected.
e. Post wash bacteria growth is instantaneous.
f. Body residues like sweat and dead skin are breeding grounds for bacteria.

Laundry washing of regular textiles leads to excess consumption of water. Moreover, huge quantities of detergents are used to launder the clothes, and this process is excessively time consuming due to long laundry wash times.

Industrial, medical and home fabric softener of interest are liquid compounds that alters the textile product's fibers by coating the fibers and making the textile product feel better to the touch (known as hand). The softening agents applied are hygroscopic or lubricating agents, which allow for the fibers to slide within the fabric structure, assisting with easier deformation and creasing of the fabric. The duration of the effect is temporary since the softeners applied during the treatment are eliminated by future machine washings.

A number of textile softener chemistries exists such as anionic and cationic surfactants, siloxane polymeric-based softeners and non-ionic surfactant softeners. The relative ease of application of the softener is seen when all that is required is to add a fabric softener to a washing machines for application during the rinse cycle. Fabric softeners work by depositing lubricating oil type chemicals on the fabric that make it feel softer, reduce static cling, and remove odors or impart a fresh fragrance.

The textile garment and hosiery industries have treated textile fabrics in the mills with antimicrobial agents to reduce the attraction and population growth of bacteria. The result is a reduction in odors generated by the bacteria on the garments and hosiery. Application of the antimicrobial agent is done by padding, spraying or foaming an ingredient or ingredients onto fabric and fusing the chemistry into the fabric (heat setting) in a heating tunnel for a more permanent treated garment.

Silver impregnation and Dimethyloctadecyl trimethoxysilane quaternaries to covalent bond to the fibers are two of the current methods.

Grey U.S. Pat. No. 4,166,794 teaches a combination product of a liquid, bleach softener composition for use in one or more of the wash, rinse and tumble dry cycles of a laundering process comprising a water soluble peroxy bleaching agent and a water soluble cationic nitrogen softener compound, at least 50% thereof being an aliphatic, quaternary ammonium compound and/or a cationic heterocyclic imide such as an imidazolinium or pyridinium compound. Grey does not mention the incorporation of antimicrobial agents to reduce micro-organism populations. The peroxy bleaching agent was for the removal of food or beverage stains.

U.S. Pat. No. 4,184,004 to Pines et al discloses fabrics namely cellulose and blends of cellulose and polyester that can be treated with the trimethoxysilyl functional groups of a terpolymer chemistry using the siloxane and an epoxy polyoxyalkylene. The results produced a soft hydrophilic fabric.

U.S. Pat. No. 4,425,372 to Baldwin reveals industrial chemical biocidal methodologies to make antimicrobial, non-leachable fabrics using Dimethyloctadecyl 3-trimethoxysilyl propyl chloride as the bactericidal and bacteriostatic chemistry. Concentrations ranging from 0.2 to 1.1 wt % are used as coatings and approved by EPA and FDA for medical devices such as drapes and pre-op fabrics. Baldwin states a 95% efficacy against bacteria in one hour but did not disclose the species.

During the past 34 years, Baldwin did not consider the evolution of bacteria within hospital settings due to horizontal gene transfer of Staphylococcus aureus to become resistant to antibiotics (methicillin) and making the MRSA more difficult to kill with the one biocide namely the dimethyloctadecyl 3-trimethoxysilyl propyl chloride. Baldwin does not disclose the addition of a textile softener and the combined synergies.

U.S. Pat. No. 4,721,511 to Kupits describes a leach resistant antimicrobial fabric namely a non woven cellulose, polyethylene, or polypropylene treated with (3-trimethoxysilyl) propyl octadecyl dimethyl ammonium chloride and an organic titanate.

Humans are exposed to bacteria in much of their environment on a 24-hour daily basis. There are beneficial species of bacteria however humans who are immune compromised can be more susceptible to infections.

In health care setting Clostridium difficile spores float in the air until nine spores combine to form a terminal loading which then allows the grouping to fall due to gravity. The result if contacted and ingested results in illness.

Therefore, a need exists for a method to reduce bacteria populations that comes in contact with the human body but is itself non-toxic, safe and that provides semi-permanent to permanent antimicrobial efficacy on linens and garments.

The anti-microbial, anti-viral, fabric softener compounds also imparts antimicrobial efficacy to the wash water during the rinse cycle and will impart a bacteriostatic environment on the washing machine interior walls.

### SUMMARY OF THU INVENTION

The invention provides a liquid antimicrobial and antiviral fabric treating composition comprising:

(a) a quaternary ammonium compound having fabric softening properties; and

(b) an antimicrobial agent having the formula I

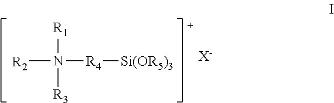

wherein R₁ is an C₁₋₃₀ alkyl group, preferably C₈₋₃₀ alkyl group, R₂ and R₃, R₄ and R₅ each independently are an C₁₋₃₀ alkyl group or hydrogen, and X is a counter ion, such as Cl⁻, Br⁻, I⁻ or CH3COO⁻.
A purpose of this invention is to expand upon the fabric softeners benefits by the addition of incorporating antimicrobial properties to the fabric during the same wash machine rinse cycle treatment. The results would reduce or kill bacteria on the fabric fibers, and reduce odors, while making the garments soft and more appealing to the touch. Antimicrobial treated fabric would affect population growth of micro-organisms on the fabric and the spreading of micro-organisms from one contact point to another contact point.

The continued evolution of micro-organisms species to generate resistant strains due to horizontal gene or RNA transfer poses a more aggressive response to antimicrobial discipline in community environmental settings such as adult care facilities as well as health care environments. Outbreaks of Methicillin Resistant Staphylococcus Aureus, Clostridium difficle, E coli and Coronaviridae and Noro virus families continue to plague hospitals, nursing homes, airlines, ocean line cruise ships and college campus settings. Even more alarming are headlines such as according to the Cancer Society that bacteria and virus may be linked to cancer and as we study the gut micro-biome we will learn more of the inter-twined chemistry of virulent endotoxins.

The invention combines well established chemistries of fabric softeners and introduces their combination into a mixture with synergistic antimicrobial agents to treat fabrics. The results are removing and/or preventing any bacterial micro-organism growth and thereby removing odor. The chemistry of a trimethoxy silane quat to impinge onto the fibers as an antimicrobial mesoporous self-assembling silane polymeric allows for a persistence efficacy of antimicrobial properties. The chemistry is heat set onto the fibers during the drying of fabrics. The compounds also treat the water and washing machine interior to include sealing gaskets preventing mold and odors.

The compositions of the present invention include a combination of substances, which have microbicidal synergistic effects. The synergistic effect reduces the need to use large quantities of biocides. Kupits does not state of killing MRSA nor does he state of additional fabric softener adds.

In the textile industry since the 1970s, Dimethyl octadecyl [(3-trimethoxysilyl) propyl]ammonium chloride has been used to treat hosiery, under garments, sports fashions and fabrics for mattress ticking. It is applied at 1.0 to 3% by weight of goods. Its claim is to remove odors produced from bacteria and is expressed as an EPA treated article (product) exemption. It has limitations to the species and time it is efficacious.

The current invention claims to make the fabrics soft, claims to remove odors, claims to remove bacteria, and claims efficacious enough to kill bacteria including MRSA.

Textile softeners include various chemistries. The imidazolinium compounds used in the present invention are Imidazolium compounds, 4,5-dihydro-1-methyl-2-nortallow alkyl-1-(2-tallow amidoethyl), Me sulfates. Tallow fatty acid or palm oil stearin are included in this dimethyl sulfate quat.

Also included are the triethanolamine fatty acid esters in the present invention, commercial mixtures of fatty acids or fatty acid methyl esters, such as those having from 10 to carbon atoms may be used as the dimethyl sulfate quat known as ester quats.

Also included are amino functional and diamino functional siloxane emulsions which bond to the fabric's fibers and synergistically combine with the dimethyloctadecyl [(3-trimethoxysilyl) propyl] ammonium chloride antimicrobial.

A primary object of the present invention is to provide a liquid fabric conditioning composition wherein treatment of the textile fabric can attain a superior soft hand feel by treating in the wash machine rinse cycle.

A further object of the invention is to provide a liquid laundry additive which is a sanitizing laundry antimicrobial capable of reducing bacteria ≥99.9% (a 3-log₁₀ reduction) both on the fabric and in the laundry water. Thereby the fabric-softener composition is capable of simultaneously and effectively mitigate bacteria and softening fabrics treatment therewith.

A still further object of the invention is to provide a liquid laundry additive composition which provides a residual self-sanitizing activity over a time period from 1 hour to 60 days. Once the fabric is treated in the rinse cycle and dried in an automatic machine washing and drying operation, the fabric will provide a reduction of greater than 99.9% in some bacteria and virus over the control count for both the fabric and laundry water within the contact time claimed.

In a further aspect, the invention includes a process of conditioning fabrics by simultaneous sanitizing and softening thereof comprising contacting said fabrics in one or more of the wash, rinse and tumble drying cycles of a laundering process with an effective sanitizing and softening amount of the composition above described.

### DETAILED DESCRIPTION OF THE PREFERRED EMBODIMENTS

The term “antimicrobial”, as used herein, refers to the capability of an article to destroy (kill), inhibit the growth or reproduction of, immobilize and/or remove microorganisms, for example bacteria, fungi, yeasts and algae.

The term “antiviral” as used herein, refers to the capability of an article to destroy (kill), inhibit the growth or reproduction of, immobilize and/or remove viruses.

The compositions of the present invention provide anti-microbial and antiviral protection to fabrics. The also provide anti-viral protection against corona-viruses and more in particular Covid 19 also known as SARS-2.

The compositions of the present invention are stable aqueous compositions which contain as the active fabric softening components cationic fabric softeners or mixtures thereof which is preferably a water-dispersible quaternary ammonium compound salt or a water dispersible alkyl imidazolinium compound salt or a water dispersible alkyl imidazolinium compound salt as hereinafter described in more detail, and or other ingredients such as fatty alcohols, nonionic ethoxylated or propoxylated alcohols also as hereinafter described.

The aqueous compositions of this invention contain as the first active fabric softening component at least one cationic fabric softener in an amount generally ranging from about 74 to 86% by weight, preferably from about 75 to 85% by weight of the total active fabric softening components (cationic fabric softener plus other ingredients), or on the basis of the total aqueous composition, the amount will be: for a concentrated formulation, from about 8 to 15% by weight, preferably from about 10 to 13%, by weight and for a ready-to-use formulation from about 2.2 to 7%, preferably from about 2.3 to 6.6%, by weight.

It is understood, of course, that a ready-to-use product is one for which the consumer is instructed to use the product at full strength (i.e. without further dilution except, of course, for the rinse water, etc.) at the specified dosage level, e.g. ¼ to ½ cup per load of wash (usually about 6 to 15 pounds). On the other hand, a concentrated formula is one for which the consumer is instructed to dilute the product (e.g. from about 1 to 4×) to make up the required dosage, e.g. ¼ to ½ cup per load of wash. Accordingly, it is clear that for the preferred concentrated formula, the consumer will have at least the perceived notion of greater economy and flexibility in use.

Softening agents are used to render fabrics or textile soft, and the terms “softening” and “softener” refer to the handle, hand, touch, or feel; this is the tactile impression given by fabrics or textiles to the hand or body and is of aesthetic and commercial importance. The cationic fabric softeners used in the present invention can be any of the commercially available and known cationic fabric softeners and preferably are of the water-dispersible quaternary ammonium compound salt or alkyl imidazolinium compound salt type including at least one, and preferably two hydrophobic groups containing at least 12 and preferably at least 14 carbon atoms.

A first preferred class of the cationic fabric softener active ingredient are the imidazolinium compounds of the formula I:

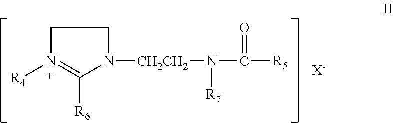

wherein R₄ is hydrogen or lower alkyl of 1 to 4 and preferably 1 to 3 and especially preferably 1 or 2 carbon atoms, R₅ is an alkyl containing from 9 to 25 carbon atoms, preferably linear higher alkyl of from about 13 to 23 and especially preferably 13 to 19 carbon atoms, R₆ is an alkyl containing from 8 to 25 carbons and preferably a substantially linear higher alkyl group of about 13 to 23, and preferably 13 to 19 carbon atoms; R₇ is hydrogen or an alkyl containing from 1 to 4 carbon atoms, preferably 1 or 2 carbon atoms, and X is as defined above.

Typical examples of the alkyl imidazolinium compounds of formula I include:
methyl-1-tallow amido-ethyl-2-tallow imidazolinium methyl sulfate,
methyl-1-oleyl amido-ethyl-2-oleyl imidazolinium methyl sulfate,
1-methyl-1-(palmitoylamido)-ethyl-2-octadecyl-4,5-dihydroimidazolinium chloride,
2-hepadecyl-1-methyl-1-(2-stearylamido)-ethyl-imidazolinium chloride,
2-lauryl-1-hydroxyethyl-1-oleyl-imidazolinium chloride,
2-tallow-1-(2-stearoyloxyethyl)-imidazoline chloride,
2-tallow-1-(2-stearoyloxyethyl)-imidazoline sulfate,
2-tallow-1-(2-stearoyloxyethyl)-imidazoline methosulfate,
2-tallow-1-methyl-3-(2-stearoylamidoethyl)-imidazoline chloride,
2-tallow-1-methyl-3-(2-stearoylamidoethyl)-imidazoline sulfate,
2-tallow-1-methyl-3-(2-stearoylamidoethyl)-imidazoline methosulfate,
2-heptadecyl-1-methyl-1-oleylamidoethyl-imidazolinium-metho-sulfate,
2-heptadecyl-1-methyl-1-(2-stearoylamido)ethyl-imidazolinium-sulfate,
2-coco-1-(2-hydroxyethyl)-1-benzyl-imidazolinium-chloride,
2-coco-1-(2-hydroxyethyl)-1-(4-chlorobutyl)-imidazolinium-chloride,
2-coco-1-(2-hydroxyethyl)-1-octadecenyl-imidazolinium-chloride,
2-tallow-1-(2-hydroxyethyl)-1-benzyl-imidazolinium-chloride,
2-tallow-1-(2-hydroxyethyl)-1-(4-chlorobutyl)-imidazolinium-chloride,
2-heptadecenyl-1-(2-hydroxyethyl)-1-(4-chlorobutyl)-imidazolinium-chloride,
2-heptadecenyl-1-(2-hydroxyethyl)-1-benzyl-imidazolinium-chloride and
2-heptadecenyl-1-(2-hydroxyethyl)-1-octadecyl-imidazolinium-chloride.

Another preferred class of the cationic softeners are the quaternary ammonium salts of the formula II:

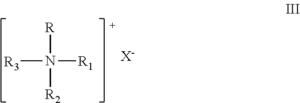

wherein R represents a hydrocarbyl group of from about 12 to 24 and preferably about 14 to 22 carbon atoms; R₁ represents lower alkyl of 1 to 4, preferably 1 to 3, carbon atoms, or a hydrocarbyl group of from 12 to 24, preferably 14 to 22, carbon atoms; R₂ and R₃ represent lower alkyl of 1 to 4, preferably 1 to 3, carbon atoms, and X represents an anion capable of imparting water solubility or dispersibility, such as halide, e.g. chloride, bromide and iodide; sulfate, methosulfate, nitrite, nitrate, phosphate, and carboxylate, e.g. acetate, adipate, propionate, phthalate, benzoate, oleate, etc.).

The hydrocarbyl groups are preferably alkyl but may be alkenyl, aryl, or aralkyl and may include various substituents or interrupting groups such as halo, amide, hydroxyl, and carboxyl substituents or interrupting functional groups and ethoxy or polyethoxy interrupting groups. In addition, one or more of the lower alkyl groups may also be substituted, for example, by an hydroxy group. Typical cationic fabric softener compounds of formula III include the following: distearyl dimethyl ammonium chloride, ditallow dimethyl ammonium chloride, dihexadecyl dimethyl ammonium chloride, distearyl dimethyl ammonium bromide, di(hydrogenated tallow) dimethyl ammonium bromide, distearyl di(isopropyl)ammonium chloride, distearyl dimethyl ammonium methosulfate and dilauryldimethylammonium chloride.

A highly preferred class of the cationic fabric softeners of formula HI are the water-insoluble compounds wherein the groups R and R₁ are C₁₄ to C₁₈, R₂ is methyl or ethyl and R₃ is methyl, ethyl, isopropyl, n-propyl, hydroxyethyl or hydroxypropyl.

The water dispersible cationic fabric softeners which can be used in the compositions of the present invention are not limited to those described above and any of the other known useful water dispersible cationic fabric softeners can be used. Furthermore, mixtures of the above mentioned cationic fabric softeners can also be used.

The anti-microbial and antiviral component of the formulation is a suitable organosilicon quaternary ammonium salt compound represented by Formula F

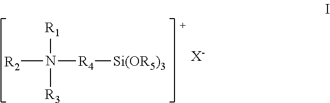

wherein R₁ is an C₁₋₃₀ alkyl group, preferably C₈₋₃₀ alkyl group, R₂ and R₃, R₄ and R₅ each independently are an C₁₋₃₀ alkyl group or hydrogen, and X is a counter ion, such as Cl⁻, Br⁻, I⁻ or CH3COO⁻.

A preferred organosilicon quaternary ammonium salt has the structural formula

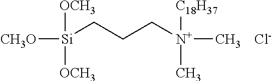

Other examples of organosilicon quaternary ammonium salt compounds for use according to the invention are 3-(triethoxysilyl)-propyl-dimethyloctadecyl ammonium chloride, 3-(tri-methoxysilyl) propyl-methyl-dioctyl ammonium chloride, 3-(trimethoxysilyl) propyl-dimethyl-decyl ammonium chloride, 3-(trimethoxysilyl)-propyl-methyldidecyl ammonium chloride, 3-(trimethoxysilyl) propyldimethyldodecyl ammonium chloride, 3-(tri-methoxysilyl)-propyl-methyl didodecyl ammonium chloride, 3-(trimethoxysilyl) propyl-dimethyltetradecyl ammonium chloride, 3-(trimethoxysilyl)-propyl-methyldihexadecyl ammonium chloride, and 3-(trimethoxy-silyl) propyl-dimethyloctadecyl ammonium chloride.

More preferably, the organosilicon quaternary ammonium salt compound is a 3-(tri-methoxysilyl) propyl-dimethyloctadecyl ammonium halide, most preferably 3-(trimethoxysilyl) propyl-dimethyloctadecyl ammonium chloride (R₁=—C₁₈H₃₇, R₂=—CH₃, R₃=—CH₃, R₄=—C₃H₆, R₅=—CH₃, and X=Cl⁻.

The concentration of the organosilicon quaternary is in the range of 1-5% by weight and more preferably in the range of 1-2.5% by weight.

Aqueous or alcoholic solutions of the compositions of the invention can be used to treat textiles, minerals, glass and many other substrates, to render the surfaces of the substrates resistant to the growth of microorganisms or viruses. Preferably, the textile fabrics treated with the compositions of the invention have free hydroxy groups on their surfaces. In this regard, it has been found that, after treatment of a substrate surface, having free hydroxy groups, with an alcohol solution of the compositions, the composition is not readily washed off or leached from the treated substrate surface by water. It is believed that this phenomenon results from the formation of covalent bonds between the silyl moiety of the silicon quaternary and the free hydroxy groups on the surface of the substrate, whereby the composition is chemically bonded to the substrate's surface.

Any substrate having free hydroxy groups on its surface can be usefully treated with the composition of the invention. Among the substrates having free hydroxy groups on their surfaces, which can be suitably bonded to the organosilicon quaternary ammonium compounds, are natural and man-made fiber fabrics, such as cotton, wool, linen, felt, acrylic, polyester, cellulose acetate, rayon, nylon and vinyl, and metals such as aluminum, stainless steel and galvanized metal, as well as wood, rubber, paper, glass and ceramic.

In the presence of moisture, this antimicrobial silicon containing quaternary ammonium agent imparts a durable, wash resistant, broad spectrum biostatic surface antimicrobial and antiviral finish to a substrate. The organosilicon quaternary ammonium compound is leach resistant, nonmigrating and is not consumed by microorganisms. It is effective against gram positive and gram negative bacteria, viruses, fungi algae, yeasts, mold, rot and mildew. The silicone quaternary ammonium salt provides durable, bacteriostatic, virucidal, fungistatic and algistatic surfaces. It can be applied to organic or inorganic surfaces as a dilute aqueous or solvent solution of 0.1-1.5 percent by weight of active ingredient. After the alkoxysilane is applied to a surface, it is chemically bonded to the substrate by condensation of the silanol groups at the surface. The pure compound is crystalline whereas methanol solutions of the compound are low viscosity, light to dark amber liquids, soluble in water, alcohols, ketones, esters, hydrocarbons and chlorinated hydrocarbons. The compound has been used in applications such as, for example, socks, filtration media, bed sheets, blankets, bedspreads, carpet, draperies, fire hose fabric materials, humidifier belts, mattress pads, health care apparel, mattress ticking, underwear, nonwoven disposable diapers, nonwoven fabrics, outerwear fabrics, nylon hosiery, vinyl paper, wallpaper, polyurethane cushions, roofing materials, sand bags, tents, tarpaulins, sails, rope, blood pressure cuffs, athletic and casual shoes, shoe insoles, shower curtains, toilet tanks, toilet seat covers, throw rugs, towels, umbrellas, upholstery fiberfill, intimate apparel, wiping cloths and medical devices such as blood pressure cuffs.

The compositions of the invention are prepared by dissolving the components in water. The resulting aqueous solutions may also include nonionic surfactants, phenoxyethanol and fragrances.

The compositions may also contain one or more optional additives selected from the group consisting of perfumes, dyes, pigments, opacifiers, optical brighteners, anti-corrosion agents, and preservatives, the amount of each additive being up to 0.5% by weight, and

In further embodiments of the invention, the starting textile material can be treated with one or more further antimicrobial agents, in particular at least one selected from the group consisting of benzalkonium chloride; benzethonium chloride; benzoxonium chloride; dequa-linium; vinylbenzyltrimethylammonium chloride; cetrimonium bromide, optionally in combination with reactive amino silicone having alkoxy groups like hydroxyl or methoxy or ethoxy groups; 2-phenylphenol, Acibenzolar, Paclobutrazol, Azoxystrobin, Epoxiconazole, Binapacryl, Iprodion, Triadimefon, Fuberidazole, Flusilazole, 2,4,6-tribromophenol, Vinciozolin, Pyrazophos, Tebuconazole, Metalaxy, Dichlofluanid, Strobilurins, Myclobutanil, Fenpropimorph with blocked isocyanate, vinylbenzyltrimethylammonium chloride, didecyldimethylammonium chloride, Fenticlor, 9-aminoacridine, polyhexamethylene biguanide, Dibromopropamidine, Chlorothalonil, Povidone-Iodine, Fenamidone, Pencycuron, cetyl pyridinium chloride, Cetrimonium, cetyl Trimethylammonium, Bupirimate, Fluopicolide, Hexachlorophene, Triclo-carban, Nitrofuran, Clioquinol, methyl-paraben, Propamocarb, cinnamaldehyde, hexamidine, and Falcarindio. The further antimicrobial agent is preferably used in an amount of 0.1 to 10% by weight, in particular in an amount of 0.1 to 5%, 0.1 to 3% or 0.1 to 1% by weight, based on the weight of the textile material.

Nonionic materials may also be incorporated in the compositions of the invention. Typical non-ionics include alkylphenoxypolyethoxyethanols having alkyl groups of about seven to eighteen carbon atoms and 6 to 60 or more oxyethylene units, such as heptyl-phenoxypolyethoxyethanols, octylphenoxynonylphenoxyethanols, dodecylphenoxypolyethoxy-ethanols, and the like; and polyethoxy ethanol derivatives of methylene-linked alkyl phenols.

The invention incorporates cationic compounds in solution to perform several functions.

### Example I

In the first set of formulations, two distinct chemistries were combined namely a cationic softening agent, 4,5 Dihydro-1-methyl-2-nortallow alkyl-1-(2-tallow amidoethyl) imidazolinium compound methyl sulfates; and a cationic biocide namely, l-Octadecanaminium-N,N-Dimethyl-N-[(3-trimethoxysilyl) propyl] chloride.

Imidazolinium DMS quaternaries are considered the best softeners on an activity basis providing exceptional softening properties in addition to providing superior static control. 4,5 Dihydro-1-methyl-2-nortallow alkyl-1-(2-tallow amidoethyl) imidazolinium compound methyl sulfates are noted for a low base odor.

The 1-Octadecanaminium-N,N-Dimethyl-N-[(3-trimethoxysilyl) propyl] chloride has a known reliability to reduce micro-organisms while binding to the textile fabric. This compound has been used on textiles and hosiery for over 40 years.

Further formulations used other biocides in addition to l-Octadecanaminium-N,N-Dimethyl-N-[(3-trimethoxysilyl) propyl] chloride to enhance the antimicrobial efficacy. The efficacy was enhanced by the addition of ammonium chloride quaternaries and polyhexa-methylene diguanide HCl.

In experimental trials 1 through 5, the compounds were mixed in a glass reactor at 50 C into deionized water.

TABLE 1
Formulations For Antimicrobial Softener

| | | TRIAL | TRIAL | TRIAL | TRIAL | TRIAL |
|---|---|---|---|---|---|---|
| ITEM | RAW MATERIAL | 1 | 2 | 3 | 4 | 5 |
| 1 | Methyl-1-Tallow Amidoethyl-2- | 12.5% | 12.5% | 12.5% | 6.2% | 3.1% |
| | Tallow Imidazolinium Methyl | | | | | |
| | Sulfates | | | | | |
| 2 | 1-Octadecaniminium-N,N-Dimethyl- | 1.04% | 1.04% | 2.2% | 2.2% | 2.2% |
| | N-[(3trimethoxysilyl) Propyl] | | | | | |
| | Chloride | | | | | |
| 3 | Deionized Water | 85.96% | 85.46% | 84.3% | 90.6% | 93.7% |
| 4 | Polyethoxylene Isodecyl Ether | — | 0.5% | 0.5% | 0.5% | 0.5% |
| 5 | Phenoxyethanol | 0.5% | 0.5% | 0.5% | 0.5% | 0.5% |
| 6 | Lemon Fragrance | — | — | — | — | — |
| | Total | 100% | 100% | 100% | 100% | 100% |

Samples of the compounds were chosen to be tested at various laboratories for antimicrobial efficacy.

The compounds were designated to the laboratories with a trade name as “Laundry Complete”. In Formulation Trial 1 a 12.5% wt of 4,5 dihydro-1-methyl-2-nortallow alkyl-1(2-tallow amidoethyl) imidazolium compounds methyl sulfates (80% wt active) fabric softener was combined with a 1.039% wt of 1-octadecaminium-N,N-dimethyl-n-[(3-tri-methoxysilyl) propyl]chloride, (72% wt active) antimicrobial in demineralized water. The raw materials were mixed at 50 C and a macro emulsion was formed.

The formulation Trial 1 was subject to microbial testing against E coli and Staphylococcus aureus. Results yielded a log 0.44 kill for E coli Table 2 and a log 0.27 kill for Staphylococcus aureus.

TABLE 2
Antimicrobial Efficacy Laundry Complete With E Coli With Formulation Trial 1
(Table 1)

| | | | AVERAGE NO | NO BACTEIA | | |
|---|---|---|---|---|---|---|
| | | BACTERIA IN | OF COLONIES | IN THE TEST | | PERCENTAGE |
| TEST | SERIAL | CONTROL | IN CONTROL | SAMPLE | LOG | KILL |
| ORGANISM | NO | CFU/ML | CFU/ML | CFU/ML | KILL | % |
| Echerichia Coli | 1 | 3.0 × 10⁷ | 3.3 × 10⁷ | 1.1 × 10⁷ | 0.44 | 63.333 |
| 8099 | | | | | | |
| Echerichia Coli | 2 | 3.3 × 10⁷ | 3.3 × 10⁷ | 1.2 × 10⁷ | 0.44 | 63.636 |
| 8099 | | | | | | |
| Echerichia Coli | 3 | 3.5 × 10⁷ | 3.3 × 10⁷ | 1.2 × 10⁷ | 0.46 | 65.714 |
| 8099 | | | | | | |

Technical Standard for Disinfection (2002 Ministry of Health P.R. China)-2.1.1.7.4 Suspension quantitative germicidal test. Test time 10 minutes.
Technical Standard for Disinfection (2002 Ministry of Health P.R. China)-2.1.1.5.5 Quantitative determination of neutralizer suspension.

TABLE 2.2
Antimicrobial Efficacy Laundry Complete With Staphylococcus Aureus Trial 1
(Table 1)

| | | | AVERAGE No. | No. BACTERIA | | |
|---|---|---|---|---|---|---|
| | | BACTERIA IN | OF COLONIES | IN THE TEST | | PERCENTAGE |
| TEST | SERIAL | CONTROL | IN CONTROL | SAMPLE | LOG | KILL |
| ORGANISM | No. | CFU/ML | CFU/ML | CFU/ML | KILL | % |
| Staphylococcus | 1 | 4.2 × 10⁷ | 4.2 × 10⁷ | 2.3 × 10⁷ | 0.26 | 45.238 |
| Aureus | | | | | | |
| ATCC 6538 | | | | | | |
| Staphylococcus | 2 | 3.9 × 10⁷ | 4.2 × 10⁷ | 2.0 × 10⁷ | 0.29 | 48.718 |
| Aureus | | | | | | |
| ATCC 6538 | | | | | | |
| Staphylococcus | 3 | 4.5 × 10⁷ | 4.2 × 10⁷ | 2.4 × 10⁷ | 0.27 | 46.667 |
| Aureus | | | | | | |
| ATCC 6538 | | | | | | |

Technical Standard for Disinfection (2002 Ministry of Health P.R. China)-2.1.1.7.4 Suspension quantitative germicidal test. Test time 10 minutes.
Technical Standard for Disinfection (2002 Ministry of Health P.R. China)-2.1.1.5.5 Quantitative determination of neutralizer suspension.

While the log kills of 0.44 and 0.27 proved the antimicrobial agent 1-octadecaminium-N,N-dimethyl-n-[(3-trimethoxysilyl) propyl]chloride at 1.0% wt had an effect, the effect was not at the log 3 range (99.9%) needed in 10 minutes.

### Example II

Formulations Trials 6 through Trial 10 from Table 3 added a mixture of ammonium chloride quaternaries to include alkyl dimethyl benzyl ammonium chloride, octadecyl dimethyl ammonium chloride, dioctyldecyl dimethyl ammonium chloride and didecyl dimethyl ammonium chloride at a 0.36 wt % to the 12.5% wt of 4,5 dihydro-1-methyl-2-nortallow alkyl-1(2-tallow amidoethyl) imidazolium compounds methyl sulfates (80% wt active) fabric softener combined with a 1.039% wt of l-octadecaminium-N,N-dimethyl-n-[(3-trimethoxysilyl) propyl]chloride, (72% wt active) antimicrobial in demineralized water.

A quantitative analysis on cotton fabric test was performed JIS L-1902. The cotton fabric was treated with Laundry Complete and held 5 days prior to commencement of JIS L-1902. The addition of 100 ml of Formulation Trial 8 from Table 3NS FOR LAUNDRYT COMPLETE per 10 Liter of water (1.0% per weight of goods) was added to the rinse cycle in a washing machine. The fabric was dried and held in constant relative humidity until dry. JIS L-1902 began. Table 4.1 shows that the efficacy for Staphylococcus aureus was log 5.8 after 20 hour duration. Table 4.2 shows the efficacy for Klebsiella Pneumoniae was log 6.3. Table 4.3 shows the efficacy for E coli was log 4.9. Table 4.4 shows the efficacy for Pseudomonas aeruginosa was log 2.2. Table 4.5 shows the efficacy for MRSA was log 6.1.

TABLE 3
FORMULATIONS FOR LAUNDRY COMPLETE

| | TRIAL 6 | TRIAL 7 | TRIAL 8 | TRIAL 9 | TRIAL 10 |
|---|---|---|---|---|---|
| CHEMICAL NAME | WT % | WT % | WT % | WT % | WT % |
| 4,5 Dihydro-1-Methyl-2-Nortallow | 12.5 | 12.5 | 12.5% | 12.5 | 6.25 |
| Alkyl-1(2-Tallow Amidoethyl) | | | | | |
| Imidazolium Compounds Methyl Sulfates | | | | | |
| (80% Active) | | | | | |
| Imidazolinium Compounds 2-(C17 And | 0 | 0 | 0 | 0 | 0 |
| C17 Unsaturated Alkyl)-1-[2-(C18 And | | | | | |
| C18 Unsaturated Amido) Ethyl]-4,5 | | | | | |
| Dihydro-1-Methyl Sulfates In Propylene | | | | | |
| Glycol | | | | | |
| 1-Octadecaminium-N,N-Dimethyl-N-[(3- | 1.039 | 1.039 | 1.039 | 1.039 | 1.039 |
| Trimethoxysilyl) Propyl]Chloride (72.0% | | | | | |
| Active) | | | | | |
| Alkyl Dimethyl Benzyl Ammonium | 0 | 0.36 | 0.36 | 0.36 | 0.36 |
| Chloride & Octyl Decyl Dimethyl | | | | | |
| Ammonium Chloride & Dioctyl | | | | | |
| Dimethyl Ammonium Chloride & | | | | | |
| Didecyl Dimethyl Ammonium Chloride | | | | | |
| Polihexanide (20% Active) | | | 0.50 | 0.50 | 0.50 |
| Deionized Water | 86.081 | 85.764 | 85.624 | 85.324 | |
| Phenoxyethanol | | | | 0.3 | 0.3 |
| Polyoxyethylene 9 Tridecyl Ether | 0.38 | 0.33 | 0.33 | 0.33 | 0.33 |
| Fragrance | | 0.007 | 0.007 | 0.007 | |

TABLE 4.1
Quantatative Method For Antimicrobial Efficacy By Absorbtion Method JIS L 1902: 2008
For Staphyloccocus Aureus Attc 6538 For Laundry Complete Table 3 Formula Trial 8

| | | DIFFERENCE OF | DIFFERENCE OF | | | |
|---|---|---|---|---|---|---|
| | | EXTREMES | EXTREMES | | | ANTI- |
| | CONCEN- | FOR 3 CONTROL | FOR 3 CONTROL | GROWTH | GROWTH | MICROBIAL |
| | TRATION | SPECIEMENS | SPECIEMENS | VALUE OF F | VALUE OF G | ACTIVITY |
| | OF | (LOG) | (LOG) | (F = LOGCₜ − | (G = LOGTₜ − | VALUE |
| HRS | INOCULUM | (CONDITION <1 | (CONDITION <2 | LOGC₀) | LOGT₀) | (A = F − G) |
| | 2.1 × 10⁵ | | | +2.5 | −3.3 | 5.8 |
| | | | | LGCₜ: +7.1 | LGTₜ: +1.3 | |
| | | | | LGC₀: +4.6 | LGT₀: +1.3 | |
| 0 HR | | <0.1 | 0.0 | | | |
| 20 HR | | <0.1 | 0.0 | | | |

TABLE 4.2
Quantatative Method For Antimicrobial Efficacy By Absorbtion Method Jis L 1902: 2008
For Klebsiella Pneumoniae ATCC 4352 For Laundry Complete Table 1 Formula Trial 2

| | | DIFFERENCE OF | DIFFERENCE OF | | | |
|---|---|---|---|---|---|---|
| | | EXTREMES | EXTREMES | | | ANTI- |
| | CONCEN- | FOR 3 CONTROL | FOR 3 CONTROL | GROWTH | GROWTH | MICROBIAL |
| | TRATION | SPECIEMENS | SPECIEMENS | VALUE OF F | VALUE OF G | ACTIVITY |
| | OF | (LOG) | (LOG) | (F = LOGCₜ − | (G = LOGTₜ − | VALUE |
| HRS | INOCULUM | (CONDITION <1 | (CONDITION <2 | LOGC₀) | LOGT₀) | (A = F − G) |
| | 1.8 × 10⁵ | | | +3.1 | −3.2 | 6.3 |
| | | | | LGCₜ: +7.6 | LGTₜ: +1.3 | |
| | | | | LGC₀: +4.5 | LGT₀: +1.3 | |
| 0 HR | | 0.1 | 0.0 | | | |
| 20 HR | | <0.1 | 0.0 | | | |

TABLE 4.3
Quantatative Method For Antimicrobial Efficacy By Absorbtion Method Jis L 1902: 2008
For Escherichia Coli ATCC 8739 For Laundry Complete Table 3 Formula Trial 8

| | | DIFFERENCE OF | DIFFERENCE OF | | | |
|---|---|---|---|---|---|---|
| | | EXTREMES | EXTREMES | | | ANTI- |
| | CONCEN- | FOR 3 CONTROL | FOR 3 CONTROL | GROWTH | GROWTH | MICROBIAL |
| | TRATION | SPECIEMENS | SPECIEMENS | VALUE OF F | VALUE OF G | ACTIVITY |
| | OF | (LOG) | (LOG) | (F = LOGCₜ − | (G = LOGTₜ − | VALUE |
| HRS | INOCULUM | (CONDITION <1 | (CONDITION <2 | LOGC₀) | LOGT₀) | (A = F − G) |
| | 2.4 × 10⁵ | | | +3.4 | −1.5 | 4.9 |
| | | | | LGCₜ: +8.0 | LGTₜ: +3.1 | |
| | | | | LGC₀: +4.6 | LGT₀: +4.1 | |
| 0 HR | | <0.1 | <0.1 | | | |
| 20 HR | | <0.1 | <0.1 | | | |

TABLE 4.4
Quantatative Method For Antimicrobial Efficacy By Absorbtion Method Jis L 1902: 2008
For Pseudomonas Aeruginosa ATCC 9027 For Laundry Complete Table 3 Formula Trial 8

| | | DIFFERENCE OF | DIFFERENCE OF | | | |
|---|---|---|---|---|---|---|
| | | EXTREMES | EXTREMES | | | ANTI- |
| | CONCEN- | FOR 3 CONTROL | FOR 3 CONTROL | GROWTH | GROWTH | MICROBIAL |
| | TRATION | SPECIEMENS | SPECIEMENS | VALUE OF F | VALUE OF G | ACTIVITY |
| | OF | (LOG) | (LOG) | (F = LOGCₜ − | (G = LOGTₜ − | VALUE |
| HRS | INOCULUM | (CONDITION <1 | (CONDITION <2 | LOGC₀) | LOGT₀) | (A = F − G) |
| | 2.4 × 10⁵ | | | +3.0 | 0.8 | 2.2 |
| | | | | LGCₜ: +7.6 | LGTₜ: +5.4 | |
| | | | | LGC₀: +4.6 | LGT₀: +4.5 | |
| 0 HR | | <0.1 | <0.1 | | | |
| 20 HR | | 0.1 | 0.1 | | | |

TABLE 4.5
Quantatative Method For Antimicrobial Efficacy By Absorbtion Method Jis L 1902: 2008
For Methicillin Resistant Staphylococcus Aureus ATCC 33591 For Laundry Complete Table 3 Formula Trial 8

| | | DIFFERENCE OF | DIFFERENCE OF | | | |
|---|---|---|---|---|---|---|
| | | EXTREMES | EXTREMES | | | ANTI- |
| | CONCEN- | FOR 3 CONTROL | FOR 3 CONTROL | GROWTH | GROWTH | MICROBIAL |
| | TRATION | SPECIEMENS | SPECIEMENS | VALUE OF F | VALUE OF G | ACTIVITY |
| | OF | (LOG) | (LOG) | (F = LOGCₜ − | (G = LOGTₜ − | VALUE |
| HRS | INOCULUM | (CONDITION <1 | (CONDITION <2 | LOGC₀) | LOGT₀) | (A = F − G) |
| | 1.5 × 10⁵ | | | +3.0 | −3.1 | 6.1 |
| | | | | LGCₜ: +7.4 | LGTₜ: +1.3 | |
| | | | | LGC₀: +4.4 | LGT₀: +2.3 | |
| 0 HR | | 0.1 | 0.1 | | | |
| 20 HR | | 0.1 | 0.0 | | | |

### Example III

In Formulations from Table 5 Trials 11 through 15, the use of a 80% Active 4,5 dihydro-1-methyl-2-nortallow alkyl-1(2-tallow amidoethyl) imidazolium compounds methyl sulfates was used at various concentrations. The compounds were mixed in 50° C. Demineralized water with and without surfactants.

Stable emulsions were made, and viscosities were suitable for the 7.5% by wt 4,5 dihydro-1-methyl-2-nortallow alkyl-1(2-tallow amidoethyl) imidazolium compounds methyl sulfates

TABLE 5
FORMULATIONS FOR LAUNDRY COMPLETE

| | TRIAL | TRIAL | TRIAL | TRIAL | TRIAL |
|---|---|---|---|---|---|
| | 11 | 12 | 13 | 14 | 15 |
| CHEMICAL NAME | Wt % | Wt % | Wt % | Wt % | Wt % |
| 4,5 Dihydro-1-Methyl-2-Nortallow Alkyl- | 11.1 | 7.5 | 6.2 | 6.2 | 6.2 |
| 1(2-Tallow Amidoethyl) Imidazolium | | | | | |
| Compounds Methyl Sulfates (80% Active) | | | | | |
| Imidazolinium Compounds 2-(C₁₇ And C₁₇ | 0 | 0 | 0 | 0 | 0 |
| Unsaturated Alkyl)-1-[2-(C₁₈ And C₁₈ | | | | | |
| Unsaturated Amido) Ethyl]-4,5 Dihydro-1- | | | | | |
| Methyl Sulfates In Propylene Glycol | | | | | |
| 1-Octadecaminium-N,N-Dimethyl-N-[(3- | 1.039 | 1.039 | 1.039 | 1.039 | 1.039 |
| Trimethoxysilyl) Propyl]Chloride (72% Wt | | | | | |
| Active) | | | | | |
| Alkyl Dimethyl Benzyl Ammonium | 0.36 | 2.25 | 2.25 | 2.25 | 2.25 |
| Chloride & Octyl Decyl Dimethyl Ammonium | | | | | |
| Chloride & Dioctyl Dimethyl Ammonium | | | | | |
| Chloride & Didecyl Dimethyl Ammonium | | | | | |
| Chloride | | | | | |
| Polihexanide | | | | 0.50 | 0.50 |
| Deionized Water | 86.864 | 90.464 | 91.864 | 92.194 | 91.371 |
| Phenoxyethanol | 0.3 | 0.3 | 0.3 | 0.3 | 0.3 |
| Polyoxyethylene Isodecyl 6 | 0.33 | 0.33 | 0.33 | | 0.33 |
| Fragrance | 0.007 | 0.007 | 0.007 | 0.007 | |

### Example IIIA

TABLE 5.1
Quantatative Method For Antimicrobial Efficacy By Absorbtion Method Iso20743: 2013
For Staphylococcus Aureus Attc 6538 For Laundry Complete Table 5 Formula Trial 15

| | | DIFFERENCE OF | DIFFERENCE OF | | | |
|---|---|---|---|---|---|---|
| | | EXTREMES | EXTREMES | | | ANTI- |
| | CONCEN- | FOR 3 CONTROL | FOR 3 TEST | GROWTH | GROWTH | MICROBIAL |
| | TRATION | SPECIEMENS | SPECIEMENS | VALUE OF F | VALUE OF G | ACTIVITY |
| | OF | (LOG) | (LOG) | (F = LOGCₜ − | (G = LOGTₜ − | VALUE |
| HRS | INOCULUM | (CONDITION <1 | (CONDITION <2 | LOGC₀) | LOGT₀) | (A = F − G) |
| | 1.4 × 10⁵ | | | 3.4 | −3.3 | 5.7 |
| | | | | LGCₜ: +7.7 | LGTₜ: +2.0 | |
| | | | | LGC₀: +4.3 | LGT₀: +4.3 | |
| 0 HR | | 0.2 | 0.0 | | | |
| 24 HR | | 0.1 | 0.0 | | | |

TABLE 5.2
Quantatative Method For Antimicrobial Efficacy By Absorbtion Method Iso 20743: 2013
For Klebsiella Pneumoniae ATCC 4352 For Laundry Complete Table 5 Formula Trial 15

| | | DIFFERENCE OF | DIFFERENCE OF | | | |
|---|---|---|---|---|---|---|
| | | EXTREMES | EXTREMES | | | ANTI- |
| | CONCEN- | FOR 3 CONTROL | FOR 3 CONTROL | GROWTH | GROWTH | MICROBIAL |
| | TRATION | SPECIEMENS | SPECIEMENS | VALUE OF F | VALUE OF G | ACTIVITY |
| | OF | (LOG) | (LOG) | (F = LOGCₜ − | (G = LOGTₜ − | VALUE |
| HRS | INOCULUM | (CONDITION <1 | (CONDITION <2 | LOGC₀) | LOGT₀) | (A = F − G) |
| | 3.1 × 10⁵ | | | +3.0 | −2.7 | 5.7 |
| | | | | LGCₜ: +7.7 | LGTₜ: +2.0 | |
| | | | | LGC₀: +4.7 | LGT₀: +4.7 | |
| 0 HR | | 0.5 | 0.0 | | | |
| 24 HR | | 0.0 | 0.0 | | | |

### Example IV

With the SARS CoV2 pandemic affecting the world, a series of experiments at the University of Alabama Huntsville Ga. under Dr Debra Moriarity were conducted with a SARS CoV-2 CDC surrogate, namely a Murine Hepatitis virus, MHV-S. MHV-S was grown in a mouse liver cell line, NCTC clone 1469. Isolated virus was incubated with a dilution of the Laundry Complete based on its use instructions, at room temperature for 15 minutes. The samples were then neutralized and passed over a dextran bead column to remove potential cytotoxic substances. Column eluates were used to inoculate NCTC done 1469 cells in a 96 well plate. After 7 days the wells were examined for cytopathological effects (CPE) in the cells and scored accordingly. The protocol followed DIN EN-14476 European Standard for Quantitative Suspension Test of Chemical Disinfectants and Antiseptics for Virucidal Activity, December 2015.

Cell culture: NCTC Clone 1469 (ATCC® CCL-9.1™) was maintained in DMEM with 4500 g/l glucose plus L-gln and 1.5 g/l sodium bicarbonate, pH 7.3, supplemented with 10% Donor Horse Serum (Biotechne, Minneapolis, Minn.) in a humidified incubator at 37° C. and 5% CO₂. Cells were passaged by scraping cells from the flask surface, centrifuging and resuspending in new growth media. 5×10⁴ cells/well were plated in DMEM+10% horse serum in a 96 well plate 24 hours before the assay and incubated as above.

Virus preparation: MHV-S (ATCC VR-766™) was used to inoculate NCTC Clone 1469 cells at a moi of about 1.0 following published procedures (Leibowitz et al., 2011). Isolated virus was stored at ˜80° C. in 1.0 ml aliquots. Virus titer was determined using the endpoint dilution procedure to obtain the TCID₅o on the NCTC Clone 0.1469 cells. Suspension Test Laundry Complete, Lot 05062020-LC-A, was diluted at a ratio of 4.69 mi to 250 ml with ultrapure water. All tests contained 0.03% bovine serum albumin (BSA) as a clean interfering substance.

Sterile tubes were set up as follows:

Tube A: Virus Titer: 0.1 ml stock virus, 0.1 ml 0.3% BSA, 0.8 ml PBS (phosphate buffered saline)

Tube B: Product Test: 0.1 ml stock vims, 0.1 ml 0.3% BSA, 0.8 ml diluted Laundry Complete

Tube C: Cytotoxicity Control: 0.1 ml DMEM+2% horse serum (DMEM-2), 0.1 ml 0.3% BSA, 0.8 ml diluted Laundry Complete

Tube D: Virus killing control: 0.1 ml stock virus, 0.1 ml 0.3% BSA, 0.2 ml 1.4% Formaldehyde

All tubes were incubated for 15 minutes at 21.8° C., 52% relative humidity. At the end of that incubation 1.0 ml of Dey/Engly Broth (DIE broth) neutralizer was added to tubes A, B and C and 0.4 ml of DIE broth was added to tube D. All tubes were place on ice. A 500 μl sample from each tube was placed on a 4.0 ml column of Sephadex LH-20 and centrifuged at 100×g at 4° C. for 10 min. The eluant was collected and serial 10-fold dilutions were made in ice-cold DMEM.

Media was removed from cells in the 96-well plate and 100 μl samples from each test condition were added to quadruplicate wells. The plates were incubated at 37° C. and 5% CO₂ for 7 days, at which time they were examined for CPE.

TABLE 6.1
Results - Test done on Jul. 3, 2020; plate scored on Jul. 10, 2020

| Dilution (Log 10) | Virus control | Test Product | Cytotoxicity | Cell Control |
|---|---|---|---|---|
| −2 | ++++ | CT | ++++ | 0000 |
| −3 | ++++ | 0++0 | 0000 | NA |
| −4 | ++++ | 0000 | 0000 | NA |
| −5 | ++++ | 0000 | 0000 | NA |
| −6 | ++++ | 0000 | 0000 | NA |
| −7 | +00+ | 0000 | 0000 | NA |
| Log10TCID₅₀ | −7.0 | −3.0 | −2.5 | NA |
| Log 10 Reduction | | 4.0 | | NA |
| % kill | | >99.99% | | NA |

+ CPE (cytopathic/cytotoxic effect) present
0 CPE (cytopathic/cytotoxic effect) not detected
N/A Not applicable
CT Cytotoxicity

A neutralizer cytotoxicity control, run separately, showed no CPE at any dilutions of the DIE broth.
Formaldehyde killed all cells to the 10⁴ dilution.
The results showed significant kill.
The virus TCID₅₀ (log 10) was determined to be 7.0
The virus TCID₅₀ (log 10) after treatment with Laundry Complete was 3.0.
The log difference between them was 4.0 indicating 99.99% of the vims was killed.

### Example V

Dimethyloctadecyl (3-trimethoxysilyl) propyl ammonium chloride is known to covalently bind to textile fabrics and will maintain a presence for up to 25 industrial washes. Dimethyloctadecyl (3-trimethoxysilyl) propyl ammonium chloride is also known to be bacteriastatic and fungistatic but not bactericidal, viricidal nor fungicidal. The Laundry Complete formulation containing ammonium chloride quats, and polyhexamethylene biguanide hcl form a polymeric nano composite matrix which imparts antiviral traits which are persistent. Continuing with the SARS CoV2, the second of a series of experiments at the University of Alabama Huntsville Ga. under Dr Debra Moriarity were conducted with a SARS CoV-2 CDC surrogate, namely a Murine Hepatitis virus, MHV-S. MHV-S was grown in a mouse liver cell line, NCTC clone 1469

Isolated virus was incubated with a piece of fabric treated with My Shield Laundry Complete (Treated on Dec. 16, 2019 and kept in a sterile container since then). Controls were untreated fabric pieces of the same size. After incubation with the virus for 15, 30 or 60 minutes the samples were neutralized with D/E broth, vortexed to remove the virus from the fabric and passed over a Sephadex LH-20 column to remove potential cytotoxic substances. Serial dilutions of the column eluates were used to inoculate NCTC clone 1469 cells in a 96 well plate to determine the TCID₅₀ for the virus at each time. After 14 days the wells were examined for cytopathological effects (CPE) in the cells and scored accordingly.

TABLE 6.2
TREATED TEXTILE WITH LAUNDRY COMPLETE AT
DAY 200 AND RESULTS AS ANTIVIRAL BIOCIDE

| Dilution | Virus, no | 15 minute** | | 30 minute** | | 60 minute** | | |
|---|---|---|---|---|---|---|---|---|
| (−Log₁₀) | cloth | Control | Treated | Control | Treated | Control | treated | Cytotoxicity |
| 2 | ++++ | ++++ | ++++ | ++++ | ++++ | ++++ | ++++ | ++++ |
| | ++++ | ++++ | ++++ | ++++ | ++++ | ++++ | ++++ | ++++ |
| 3 | ++++ | ++++ | ++++ | ++++ | 0+++ | ++++ | ++++ | 0000 |
| | ++++ | ++++ | ++++ | ++++ | ++++ | ++++ | ++++ | 0000 |
| 4 | ++++ | ++++ | ++00 | +0++ | 0000 | ++++ | +00+ | 0000 |
| | ++++ | ++0+ | 0000 | +0++ | +++0 | ++++ | 0+0+ | 0000 |
| 5 | 000+ | 0+00 | 00+0 | ++00 | 0000 | +0++ | 000+ | 0000 |
| | 00+0 | 000+ | 0000 | ++0+ | 00+0 | ++0+ | 0000 | 0000 |
| 6 | 0000 | 0000 | 0000 | 0000 | 0000 | 0++0 | 0000 | ND |
| | 0000 | 0000 | 00+0 | 0000 | 0000 | +0+0 | 0000 | |
| 7 | 0000 | 0+00 | 0000 | 0000 | 0000 | 0+00 | 0000 | ND |
| | 0000 | 0000 | 0000 | 00+0 | 0000 | 0000 | 0000 | |
| −Log₁₀ | 4.67 | 4.44 | 3.67 | 5.20 | 3.75 | 6.00 | 4.00 | 2.50 |
| TCID₅₀/ml | | | | | | | | |
| Log | | | ≥0.77 | | ≥1.45 | | ≥2.0 | NA |
| difference* | | | | | | | | |
| % kill | | | ≥83.2% | | ≥96.5 | | ≥99% | |

+ = CPE
0 = live cells still
visible ND = not determined
*between control and treated fabric coupon for each time point
**time virus was exposed to the fabric coupon

### Results

The virus TCID₅₀ (log 10) was determined to be 4.67
Cytotoxicity was only observed at 10⁻² dilution.
A neutralizer cytotoxicity control, run separately, showed no CPE at any dilutions of the D/E broth.
Formaldehyde killed all cells to the 10⁻² dilution.
Most significantly, the persistence of the virucidal activity of the My Shield Laundry Complete was evident even after more than 200 days with at least 99% kill of virus after only an hour of contact with the fabric, and nearly 97% kill after only 30 min.

### Example VI

In a further experiment with actual wild strain SARS CoV-2 done in a BSL3 laboratory, a cotton textile product treated with Laundry Complete on Feb. 6, 2021. The sample was inoculated and tested 53 days later on Apr. 5-9, 2021.

A test suspension of the SARS-CoV-2 virus was inoculated onto My-Shield Laundry Complete coated fabric #06302020-LC-A (100% cotton) and its respective control fabric (100% cotton) surface.

The surfaces loaded with virus inoculum were maintained at specified temperature (25° C.±1° C.) for a contact period of 2 hours. At the end of the contact time remaining infectious virus particles were recovered individually from onto My-shield Laundry Complete #06302020-LC-A coated fabric specimen and its respective control fabric specimen by washing the surfaces followed by vortex and agitation in neutralizing medium. Quantification of recovered surviving organisms (infectious virus particles) was done by plaque assay. As prescribed in guideline assay was performed in triplicate using 3 test specimen for each step.

TABLE 7.1
VIRUS DATA

| | |
|---|---|
| Realm | Riboviria |
| Order | Nidovirales |
| Family | Coronaviridae |
| Genus | Betacoronavirus |
| Species | COVID-19 |
| NCBI Accession number | MT416726[MB1] |
| for virus isolate | |

TABLE 7.2
TEST OUTLINE

| | |
|---|---|
| Guideline referred | ISO 18184 (Textiles: Determination |
| | of Antiviral activity of textile products) |
| Details of the specimen | My-shield Laundry Complete coated |
| | fabric Lot #06302020-LC-A (100% |
| | cotton) and control fabric (100% cotton) |
| | of size 20 mm X 20 mm, 0.4 g |
| Date of coating of test sample | 6^{th} Feb. 2021 |
| as provided by Zeta Tech | |
| Assay performed on | 53 days after treatment My-shield |
| | Laundry Complete onto the fabric |
| | specimen |
| Specimen storage condition | Ambient |
| Virus used for testing | COVID 19 (SARS-CoV-2) P6 |
| Host Cell line used for testing | Vero cell line Pl56 |
| Volume of test inoculum used | 200 μI (0.7 × 10⁵ PFU/ml) |
| Test Concentrations | RTU (150 ml per 4.5 kg fabric in |
| | standard wash) refer CoAProduct Safety |
| | Labs # 53706 |
| Test Temperature | 25° C. ± 1° C. |
| Temperature of | 37° C. |
| incubation in plaque | |
| assay | |
| Contact time | 2 hours |
| Stability and appearance of the | 200 μl of the virus specimen was |
| specimen during the test | absorbed over a period of 2 hours in test |
| | and control specimen upper layer only. |
| Neutralizer used | Ice cold 2% Minimal Essential media |

My-Shield Laundry Complete Lot #06302020-LC-A coated fabric has exhibited antiviral activity value Mv of 2,643 against SARS-CoV2 virus after 2 hours of contact time when tested after 53 days applying coating onto the fabric (100% cotton) specimen.

The antiviral efficacy of My-shield Laundry Complete coated fabric Lot #06302020-LC-A tested after 53 days of coating onto the specimen (100% cotton fabric) has a GOOD EFFECT’ as per the antiviral performance standard against SARS-CoV2.

The results indicates that My-shield Laundry Complete treated fabric specimen Lot #06302020-LC-A exhibited virucidal activity against SARS-CoV2 virus. No live infectious SARS-CoV-2 virus particles were retained on My-shield Laundry Complete treated fabric specimen Lot #06 A panel than that of respective untreated control specimen after contact time of 2 hours.

TABLE 7.3
DATA SUMMARY

| | | | | | | | | V_{P} | | | |
|---|---|---|---|---|---|---|---|---|---|---|---|
| | Number of plaques recovered | | | | | | | (PFU/ | | M | Mν |
| | Specimen 1 | | Specimen 2 | | Specimen 3 | | z | vial) | | value | value |
| Untreated A | 25 | 21 | 22 | 25 | 13 | 9 | 19.166 | Vb | 19166 | 0.36B | 2.643 |
| Treated A# | ND | ND | ND | ND | ND | ND | 1# | Ve | 1000 | | |
| Control A | 49 | 48 | 28 | 25 | 59 | 55 | 44 | Va | 44000 | | |

A Dilution Factor: 10;
#As the sample is cytotoxic at 10⁻¹, the plaque were not determined. Since no plaques were observed in 10⁻² dilution, following ISO18184 guideline the number of plaques is recorded as ‘1’ Logarithm reduction value of infective titre of control specimen should be ≤1.0.

The contents of all references cited in the instant specifications and all cited references in each of those references are incorporated in their entirety by reference herein as if those references were each individually denoted in the text.

While the many embodiments of the invention have been disclosed above and include presently preferred embodiments, many other embodiments and variations are possible within the scope of the present disclosure and in the appended claims that follow. Accordingly, the details of the preferred embodiments and examples provided are not to be construed as limiting. It is to be understood that the terms used herein are merely descriptive rather than limiting and that various changes, numerous equivalents may be made without departing from the spirit or scope of the claimed invention.

What is claimed is:

1. A liquid antimicrobial and antiviral fabric treating composition comprising:
(a) a quaternary ammonium compound having fabric softening properties; and
(b) an antimicrobial agent having the formula I 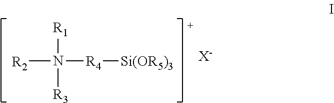
wherein R₁ is an C₁₋₃₀ alkyl group, preferably C₈₋₃₀ alkyl group, R₂ and R₃, R₄ and R₅ each independently are an C₁₋₃₀ alkyl group or hydrogen, and X is a counter ion, such as Cl⁻, Br⁻, I⁻ or CH3COO⁻.

2. The liquid composition of claim 1, wherein the quaternary ammonium fabric softener is a compound of the formula II: 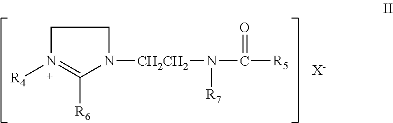
wherein R₄ is hydrogen or lower alkyl of 1 to 4 and preferably 1 to 3 and especially preferably 1 or 2 carbon atoms, R₅ is an alkyl containing from 9 to 25 carbon atoms, preferably linear higher alkyl of from about 13 to 23 and especially preferably 13 to 19 carbon atoms, R₆ is an alkyl containing from 8 to 25 carbons and preferably a substantially linear higher alkyl group of about 13 to 23, and preferably 13 to 19 carbon atoms; R₇ is hydrogen or an alkyl containing from 1 to 4 carbon atoms, preferably 1 or 2 carbon atoms, and X is as defined above.

3. The liquid composition of claim 2, wherein said quaternary ammonium fabric softener is selected from the group consisting of:
methyl-1-tallow amido-ethyl-2-tallow imidazolinium methyl sulfate,
methyl-1-oleyl amido-ethyl-2-oleyl imidazolinium methyl sulfate,
1-methyl-1-(palmitoylamido)-ethyl-2-octadecyl-4,5-dihydroimidazolinium chloride,
2-hepadecyl-1-methyl-1-(2-stearylamido)-ethyl-imidazolinium chloride,
2-lauryl-1-hydroxyethyl-1-oleyl-imidazolinium chloride,
2-tallow-1-(2-stearoyloxyethyl)-imidazoline chloride,
2-tallow-1-(2-stearoyloxyethyl)-imidazoline sulfate,
2-tallow-1-(2-stearoyloxyethyl)-imidazoline methosulfate,
2-tallow-1-methyl-3-(2-stearoylamidoethyl)-imidazoline chloride,
2-tallow-1-methyl-3-(2-stearoylamidoethyl)-imidazoline sulfate,
2-tallow-1-methyl-3-(2-stearoylamidoethyl)-imidazoline methosulfate,
2-heptadecyl-1-methyl-1-oleylamidoethyl-imidazolinium-metho-sulfate,
2-heptadecyl-1-methyl-1-(2-stearoylamido)ethyl-imidazolinium-sulfate,
2-coco-1-(2-hydroxyethyl)-1-benzyl-imidazolinium-chloride,
2-coco-1-(2-hydroxyethyl)-1-(4-chlorobutyl)-imidazolinium-chloride,
2-coco-1-(2-hydroxyethyl)-1-octadecenyl-imidazolinium-chloride,
2-tallow-1-(2-hydroxyethyl)-1-benzyl-imidazolinium-chloride,
2-tallow-1-(2-hydroxyethyl)-1-(4-chlorobutyl)-imidazolinium-chloride,
2-heptadecenyl-1-(2-hydroxyethyl)-1-(4-chlorobutyl)-imidazolinium-chloride,
2-heptadecenyl-1-(2-hydroxyethyl)-1-benzyl-imidazolinium-chloride and
2-heptadecenyl-1-(2-hydroxyethyl)-1-octadecyl-imidazolinium-chloride.

4. The liquid composition of claim 1, wherein the quaternary ammonium fabric softener is a compound of the formula HI: 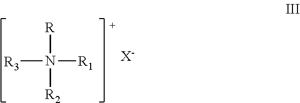
wherein R represents a hydrocarbyl group of from about 12 to 24 and preferably about 14 to 22 carbon atoms; R₁ represents lower alkyl of 1 to 4, preferably 1 to 3, carbon atoms, or a hydrocarbyl group of from 12 to 24, preferably 14 to 22, carbon atoms; R₂ and R₃ represent lower alkyl of 1 to 4, preferably 1 to 3, carbon atoms, and X represents an anion capable of imparting water solubility or dispersibility, such as halide, e.g. chloride, bromide and iodide; sulfate, methosulfate, nitrite, nitrate, phosphate, and carboxylate, e.g. acetate, adipate, propionate, phthalate, benzoate and oleate.

5. The liquid composition of claim 4, wherein said quaternary ammonium fabric softener is selected from the group consisting of:
distearyl dimethyl ammonium chloride,
ditallow dimethyl ammonium chloride,
dihexadecyl dimethyl ammonium chloride,
distearyl dimethyl ammonium bromide,
di(hydrogenated tallow) dimethyl ammonium bromide,
distearyl di(isopropyl)ammonium chloride,
distearyl dimethyl ammonium methosulfate
and dilauryldimethylammonium chloride.

6. The liquid composition of claim 1, wherein the antimicrobial agent of formula I is selected from the group consisting of:
3-(triethoxysilyl)-propyl-dimethyloctadecyl ammonium chloride,
3-(trimethoxysilyl)-propyl-methyl-dioctyl ammonium chloride,
3-(trimethoxysilyl)-propyl-dimethyldecyl ammonium chloride,
3-(trimethoxysilyl)-propyl-methyldidecyl ammonium chloride,
3-(trimethoxysilyl)-propyl-dimethyldodecyl ammonium chloride,
3-(trimethoxysilyl)-propyl-methyldidodecyl ammonium chloride,
3-(trimethoxysilyl)-propyl-dimethyltetradecyl ammonium chloride,
3-(trimethoxysilyl)-propyl-methyldihexadecyl ammonium chloride, and
3-(trimethoxysilyl)-propyl-dimethyloctadecyl ammonium chloride.

7. The liquid composition of claim 1, wherein said composition can be applied on garments in textile mills or in the home washing machine, or rinse cycle.

8. The liquid composition of claim 1, wherein said composition provides a softened hand feel on the textile garment.

9. The liquid composition of claim 1, wherein said composition removes and reduces odors on the textile garment.

10. The liquid composition of claim 1, wherein said composition provides the efficacy of removal or reduction in microorganism population on the garment.

11. The liquid composition of claim 1, wherein said composition once infused into the garments fibers provides persistent efficacy as an antimicrobial and anti-viral.

12. The liquid composition of claim 1, wherein said composition is efficacious against E coli, Staphylococcus aureus, Pseudomonas aeruginosa, Klebsiella pneumoniae and Methicillin resistant Staphylococcus aureus, Influenza, Coronaviridae and Noro virus families

13. The liquid composition of claim 1, wherein said composition is non-allergenic on garments and clothing.

14. The liquid composition of claim 1, wherein said composition is applied on textile substrates selected from the group consisting of cotton, polyester blends, and polyether-polyurea copolymers such as spandex, lycra or elastane.

15. The liquid composition of claim 1, wherein said composition provides antistatic properties to the textile garment.

16. The liquid composition of claim 1, wherein said composition provides hospital linens and uniforms with antimicrobial properties to protect the hospital care worker.

17. The liquid composition of claim 1, further including polyhexylmethylene biguanide HCl.

18. The liquid composition of claim 1, wherein said composition further includes a nonionic surfactant.

19. The liquid composition of claim 1, wherein said composition further includes a preservative.

20. The liquid composition of claim 1, wherein said composition when applied to surgical masks prevents microorganisms from traveling through the textile filter due to cationic charge and van der Walls attraction.

21. The liquid composition of claim 1, wherein said composition can be applied to treat medical bandages, gauze, pre-op non-woven, cotton gloves and surgical disposable textiles and cellulosic paper barriers.

22. A method for providing antimicrobial and antiviral properties to a fabric, said method comprising contacting said fabric with the composition of claim 1.
